(19) United States
(12) Patent Application Publication
(10) Pub. No.: US 2021/0316024 A1
(43) Pub. Date: Oct. 14, 2021

(54) UVC SANITATION DEVICE AND SYSTEM FOR FOOTWEAR AND APPAREL AND RELATED METHODS

(71) Applicant: Jeff Green, Beverly Hills, CA (US)

(72) Inventor: Jeff Green, Beverly Hills, CA (US)

(21) Appl. No.: 17/175,177

(22) Filed: Feb. 12, 2021

Related U.S. Application Data
(60) Provisional application No. 63/081,497, filed on Sep. 22, 2020.
(60) Provisional application No. 63/008,394, filed on Apr. 10, 2020.

(51) Int. Cl. A61L 2/10; A61L 2/24

(52) U.S. Cl. CPC A61L 2/10; A61L 2202/14; A61L 2202/11; A61L 2/24

(57) ABSTRACT

A footwear sanitation station with UVC emitters to radiate footwear positioned on supporting grates through which UVC radiation can irradiate outsoles of the footwear. The station includes one or more different types of sensors, a power indicator, a sanitize indicator, a power source, and a a controller with a processor allowing the station to operate in different modes, including a Stand-By Mode and a Sanitize Mode, and to activate the power indicator and the sanitize indicator to provide cues to a user of the Mode of operation.

### CROSS-REFERENCE TO RELATED APPLICATIONS

This application claims priority to and the benefit of U.S. Provisional Patent Application No. 63/081,497, filed Sep. 22, 2020, entitled UVC SANITATION DEVICE AND SYSTEM FOR FOOTWEAR AND APPAREL AND RELATED METHODS, and U.S. Provisional Patent Application No. 63/008,394, filed Apr. 10, 2020, entitled UVC INTEGRATED MAT AND RELATED SANITATION SYSTEM AND METHODS, the entire content of both of which is incorporated herein by reference.

### BACKGROUND

### Field

Some embodiments of the present disclosure relate to sanitation of goods, including apparel, by irradiation, in particular by UV irradiation of clothing and footwear.

### Description of the Related Art

With a wavelength ranging between about 200 and 300 nanometers ultraviolet light is part of the electromagnetic spectrum with wavelengths shorter than visible light but longer than X-rays. The most common form of UV radiation is sunlight, which produces three main types of UV rays: UVA, UVB and UVC. Most of the UV rays reaching the Earth's surface is UVA and a small amount of UVB.

With a wavelength ranging between about 240 and 280 nanometers that is strongly absorbed by nucleic acids, UVC is effective to kill or inactivate microorganisms, such as bacteria, viruses, mold and other pathogens, by destroying nucleic acids and disrupting their DNA. The application of UVC for disinfection has been practiced since the mid-20^{th} century. It has been used primarily in medical sanitation and sterile work facilities. Moreover, a key benefit to UV sanitation is that it requires no chemicals, so there is little risk posed to people with allergies or sensitivities.

Effectiveness of UV irradiation depends on a number of factors such as length of exposure and intensity of radiation. Line of sight exposure is another factor that affects effectiveness of UV irradiation. Thus, when designing equipment, obstacles that block the UV light should be minimized.

Germs are ubiquitous. They are on people's skin, clothing and shoes and because bacteria, viruses and other pathogens can survive on surfaces for hours, if not days, germs can be transported into the home on clothing and shoes. For example, tuberculosis can live for several days, HIV can live only for moments. Common cold viruses are hardier. E coli may live for a few hours to a day, whereas norovirus can live for days or weeks. Notably, anthrax can live for years. Therefore, it is not only hospitals or other medical establishments that can benefit from UVC sanitation but any location where people and their clothing and footwear frequent, including homes and residences. Shoes, in particular, can carry a whole host of germs. If shoes are worn through a city, sidewalks can be covered with animal droppings and human cough-ups. Public bathrooms may also expose shoes to all sorts of microbes. These germs and pathogens can easily hitch a ride on shoe bottoms and easily rub off on floors indoors.

In hospital settings, UVC sanitation may be accomplished through lighting fixtures equipped with UVC emitters to irradiate hospital rooms in periods of room vacancy. However, only those objects and surfaces in a direct line of sight of such fixed UVC irradiation are exposed to sanitation. UVC sanitation is now also being applied in hotel and other hospitality settings through the use of “UVC wands.” These are hand-held devices that are used to sanitize smaller surfaces such as TV remotes, desks and chairs. More recent applications of UVC sanitation include portable stands or cases that are adapted to receive mobile phones and smaller objects such as eyeglasses or toys.

Whereas earlier UVC sanitation devices used halogen bulbs, the industry and technology are moving toward LEDs. In comparison to LEDs, halogen bulbs require longer start-ups, utilize significantly more energy for the same quality of illumination, are less tolerant of vibrations and shock, and have shorter life spans. While LEDs have been more costly to purchase than halogens, the purchase price of LEDs has decreased substantially thanks to sharp cost reductions and performance improvements. In particular, the price of UVC LEDs has dropped in recent times. Fueled by worldwide concerns over the recent COVID-19 pandemic, demand for UVC LEDs will increase substantially, if not exponentially.

Accordingly, there is a desire, if not a need, for a UV irradiation device and system for irradiating apparel, such as clothing and shoes, that are adapted for use in homes and residences.

The above information is only for enhancement of understanding of the background of embodiments of the present disclosure, and therefore may contain information that does not form the prior art.

### SUMMARY

Some embodiments of the present disclosure provide an integrated mat comprising a mat body, a plurality of UVC emitters embedded in the mat body, an electrical circuit with an electrical switch, and a first activation sensor and a second activation sensor configured to sense presence of a user to generate a first signal and a second signal respectively, wherein the electrical switch is configured to be responsive to said first and second signals in activating the UVC emitters.

In some embodiments, the first and second activation sensors may be any combination of the following: motion sensor, proximity sensor, weight sensor and light sensor.

In some embodiments, the integrated mat includes a power supply.

In some embodiments, the power supply may be a DC power supply or an AC power supply.

In some embodiments, the power supply may be a single-use battery or a rechargeable battery.

In some embodiments, the electrical switch includes a FET switch.

In some embodiments, the integrated mat includes an electrical circuit configured to condition the supply of power to the UVC emitters solely on the occurrence of activation of both the first and second activation sensors.

In some embodiments, the integrated mat includes an electrical circuit configured to condition the supply of power to the UVC emitters solely on the simultaneous occurrence of activation of both the first and second activation sensors.

In some embodiments, the integrated mat includes a controller configured to control a duration of activation of the UVC emitters.

In some embodiments, the integrated mat includes a controller configured to control an intensity of emission of the UVC emitters.

In some embodiments, the integrated mat includes visible indicia configured to identify region on a surface of the mat for placement of a user's footwear.

In some embodiments, the integrated mat includes a first layer with openings, each opening configured to allow radiation from a respective UVC emitter to reach above an outer surface of the mat.

In some embodiments, the integrated mat includes an indicator configured to provide a signal to a user of activation of the UVC emitters.

In some embodiments, the integrated mat includes a controller with a microprocessor configured to control irradiation parameters of the UVC emitter.

In some embodiments, the irradiation parameters include duration of activation of UVC emitter.

In some embodiments, the irradiation parameters intensity of irradiation of UVC emitter.

In some embodiments, the irradiation parameters depend on proximity of a detected user.

In some embodiments, an array includes a plurality of integrated mats configured to connect in forming a generally contiguous surface to receive multiple footwear.

In some embodiments, an apparel sanitation system, includes an integrated mat configured to treat footwear and a remote UVC emitter configured to treat clothing.

In some embodiments, the UVC emitter is configured for fixed mounting on a wall or ceiling.

In some embodiments, the UVC emitter is configured as a hand-held device.

In some embodiments, a footwear sanitation station includes a housing with at least one grate configured to support footwear, a base tray configured to support the at least one grate, a plurality of UVC emitters configured to radiate the footwear supported on the at least one grate, a sensor, a power indicator, a sanitize indicator, a power source, and a controller with a processor configured to: operate the station in two modes, including a Stand-By Mode and a Sanitize Mode; and activate the power indicator and the sanitize indicator to provide cues to a user of the Mode of operation.

In some embodiments, the sensor includes a pressure sensor configured to detect the presence of an object by its weight on the platform grate.

In some embodiments, the power indicator includes a light indicator, for example, an LED, to provide a visual cue to the user of the operation of the station in the Stand-By Mode.

In some embodiments, the station includes a power switch.

In some embodiments, the station includes an edge guard configured to extend around a periphery of a respective grate.

In some embodiments, the base tray includes a well configured to receive a respective grate.

In some embodiments, each well has a plurality of ridges configured to support the grate.

In some embodiments, the well has a predetermined depth.

In some embodiments, the processor is configured to energize the UVC emitters provided the weight detected by the sensor exceeds a predetermined threshold weight.

In some embodiments, the processor is configured to activate the power indicator and the sanitizer indicator in predetermined sequences with activation of the UVC emitters as a safety measure.

In some embodiments, the processor is configured to extend time duration of the Sanitize Mode.

In some embodiments, the power source is rechargeable.

In some embodiments, the power source is rechargeable via a USB socket.

In some embodiments, a footwear sanitation device includes a base tray with at least one well, each well having at least one ridge, at least one grate each configured to be received in a respective well and supported by the ridge in the respective well, and at least one UVC emitter situated in a respective well and each configured to direct its radiation toward the grate.

In some embodiments, each well includes subwell, each of which includes at least one UVC emitter.

In some embodiments, each well includes a plurality of UVC emitters arranged in an alternating pattern.

In some embodiments, the alternating pattern includes a 2-1-2 pattern.

In some embodiments, the alternating pattern includes 1-2-1-2-1 pattern.

In some embodiments, each well includes a plurality of UVC emitters arranged in a nonalternating pattern.

In some embodiments, the nonalternating pattern includes 1-1-1-1-1 pattern.

In some embodiments, the nonalternating pattern includes 2-2-2-2-2 pattern.

In some embodiments, the footwear sanitation device further includes a rechargeable battery.

In some embodiments, the footwear sanitation device includes a tempered glass panel supported on the base tray, the tempered glass having a cutout portion surrounding the grate.

In some embodiments, the footwear sanitation device includes an edge guard for each well, each edge guard configured to extend around a peripheral edge of a grate in each well.

In some embodiments, a footwear sanitation system includes a base tray with at least one well, each well having at least one ridge, at least one grate configured to be received in a respective well and supported by the ridge in the respective well, at least one UVC emitter situated in the well and configured to direct its radiation toward the grate, a power source, a pressure sensor, a power indicator, a sanitate indicator, and processor configured to operate the system between at least a standby mode and a sanitate mode, wherein the standby mode includes energizing the power indicator, and energizing the pressure sensor to prepare for detection of a load, and wherein the sanitate mode includes energizing the sanitate indicator, and energizing the UVC emitter.

In some embodiments, the processor is configured to energize the UVC emitter for a predetermined duration.

In some embodiments, the processor is configured to deactivate the sanitate indicator before activating the sanitate indicator.

In some embodiments, the processor is configured to determine whether the load exceeds a predetermined threshold load.

In some embodiments, the processor is configured to operate in the sanitate mode solely when the load exceeds the predetermined threshold load.

In some embodiments, the processor is configured to selectively continue energizing the UVC emitter when the predetermined duration has been exceeded.

In some embodiments, the processor is configured to continue energizing the UVC emitter when the predetermined duration has been exceeded by a predetermined count.

In some embodiments, the UVC emitter has a radiation radius between about 50-60 mm.

In some embodiments, a footwear sanitation device includes a base tray with a pair of wells, each well having a plurality ridges configured to create subwells within the wells, at least one grate each configured to be received in a respective well and supported by the ridges in the respective well, and at least one UVC emitter each situated in a respective subwell and configured to direct its radiation toward the grate, each subwell defined by one dimension parallel with the ridges, each UVC emitter having a radiation radius that is less than the one dimension.

In some embodiments, the UVC emitters are arranged in a linear pattern.

In some embodiments, the UVC emitters arranged in the linear pattern are aligned along a common axis generally perpendicular to the ridges.

In some embodiments, each UVC emitter is situated generally at a center of each subwell.

In some embodiments, the UVC emitters are arranged in a nonlinear pattern.

In some embodiments, the subwells have a generally common shape.

In some embodiments, the subwells have a generally common size.

In some embodiments, the station has a longitudinal axis and a lateral axis generally perpendicular to the longitudinal axis, wherein the ridges and the one dimension are generally parallel with the lateral axis.

In some embodiments, the footwear sanitation device further includes a rechargeable battery.

In some embodiments, the footwear sanitation device includes a tempered glass panel supported on the base tray, the tempered glass having a cutout portion surrounding the grate.

In some embodiments, the footwear sanitation device includes an edge guard for each well, each edge guard configured to extend around a peripheral edge of a grate in each well.

### BRIEF DESCRIPTION OF THE DRAWINGS

Some embodiments can be understood in more detail from the following description taken in conjunction with the accompanying drawings, in which:

FIG. 1 is a top plan view of an integrated mat, according to an embodiment of the present invention.

FIG. 2 is a partial side cross-sectional view of the integrated mat of FIG. 1.

FIG. 2A is a side view of a view angle of a UVC emitter, according to an embodiment.

FIG. 2B is a bottom plan view of a footwear outsole with irradiated regions, according to an embodiment.

FIG. 3 is a block diagram of the integrated mat, according to an embodiment.

FIG. 4A is a circuit diagram of the integrated mat, according to an embodiment.

FIG. 4B is a circuit diagram of the integrated mat, according to an embodiment.

FIG. 5 is a top plan view of an array of integrated mats, according to an embodiment of the invention.

FIG. 6 is a flow chart of acts taken by a controller, according to an embodiment.

FIG. 7 is a perspective view of an apparel sanitation system, according to an embodiment.

FIG. 8 is a top plan view of a footwear sanitation device, according to an embodiment.

FIG. 9A is an exploded perspective view of the footwear sanitation device of FIG. 8.

FIG. 9B is a top plan schematic of a base panel with a first alternating UVC emitter configuration, according to an embodiment.

FIG. 9C is a top plan schematic of a base panel with a second alternating UVC emitter configuration, according to another embodiment.

FIG. 9D is a top plan schematic of a base panel with a third alternating UVC emitter configuration, according to another embodiment.

FIG. 9E is a top plan schematic of a base panel with a fourth alternating UVC emitter configuration, according to another embodiment.

FIG. 9F is a top plan schematic of a base panel with a first nonalternating UVC emitter configuration, according to another embodiment.

FIG. 9G is a top plan schematic of a base panel with a second nonalternating UVC emitter configuration, according to another embodiment.

FIG. 10 is a block diagram of electrical components of the device of FIG. 8, according to an embodiment.

FIG. 11 is a flow diagram illustrating operation of the electrical components of FIG. 10, according to an embodiment.

FIG. 12 is a flow diagram illustrating operation of the electrical components of FIG. 10, according to another embodiment.

### DETAILED DESCRIPTION

Features of the inventive concept and methods of accomplishing the same may be understood more readily by reference to the following detailed description of embodiments and the accompanying drawings. Hereinafter, embodiments will be described in more detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. The drawings are not necessarily to scale and the relative sizes of elements, layers, and regions shown may be exaggerated for clarity.

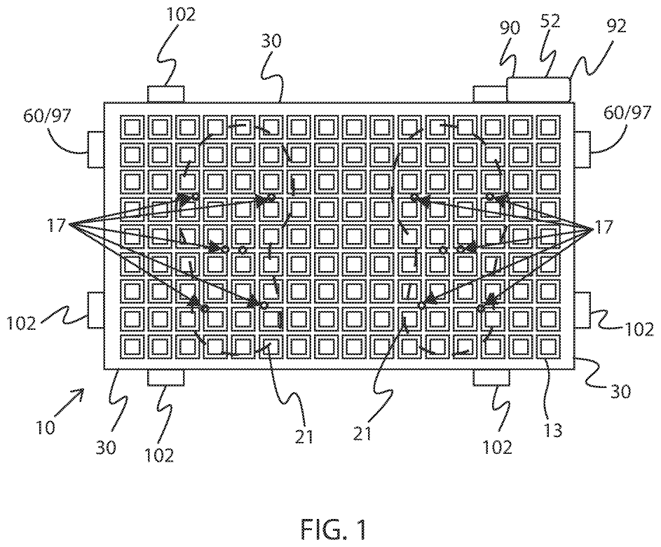

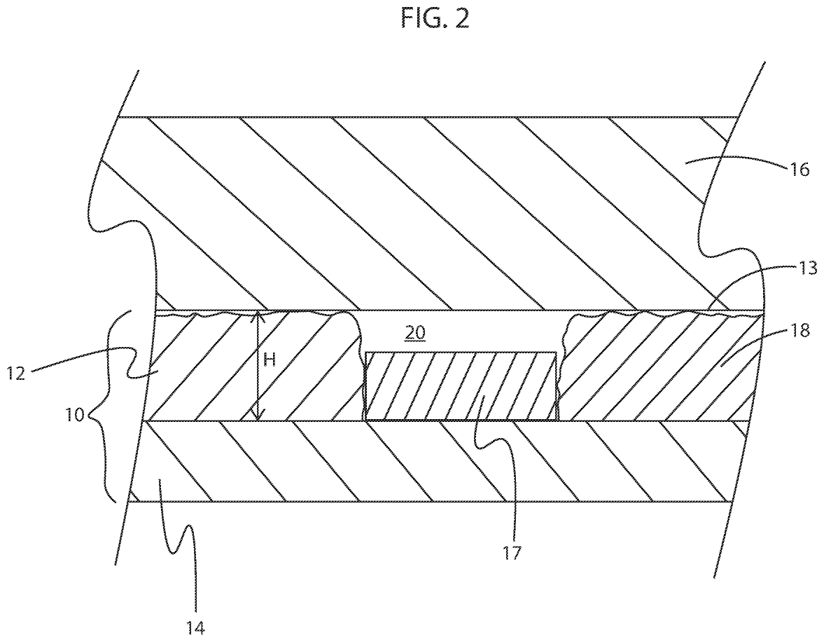

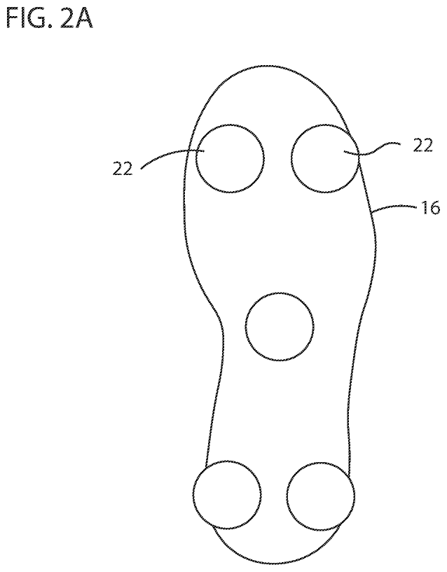

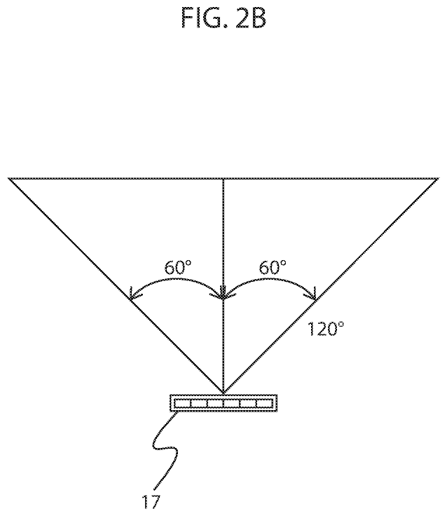

Embodiments of the present disclosure, as shown in FIG. 1 and FIG. 2, provide an integrated floor mat 10 having at mat body having least a first or top layer 12, a second or bottom layer 14, and a plurality of UVC emitters 17, for example, UVC LEDs, embedded in the mat 10. The first and second layers are stacked and affixed to each other. The top layer 12 has an outwardly-facing upper surface layer 13 configured to be stepped upon by a user and for making contact with an outsole 16 of footwear 15 worn by a user. In some embodiments, the upper surface layer 13 includes friction-inducing pile 18 (e.g., fibers or “blades”) adapted for contact with the outsole 16, including abrasive brushing of the sole against the pile when the user shuffles his/her feet on the mat 10. The pile 18 is configured with a height H that provides a predetermined, generally elastic or resilient thickness in top layer 12 that ranges between about 0.10 inch and 2.0 inches, preferably between about 0.20 inch and 1.5 inches, so that the pile is mostly resistive to a compression load up to about 250 lbs, such as when stepped upon by a user whether the user is a child or an adult. The pile 18 is fixedly attached to second layer 14 so that the user can brush the outsole of the footwear against the pile to remove dirt and debris.

A plurality of holes or openings 20 are formed in the first layer, with each opening 20 corresponding to and in alignment with a respective UVC emitter such that emission from the emitter can radiate past the pile 18 of the first layer 12 and reach above the first layer to the outsole 16 of the footwear in a direct line-of-sight pathway. The plurality of UVC emitters 17 can be arranged in any suitable or desirable pattern. In some embodiments, as illustrated in FIG. 1, the UVC emission emitters 17 are arranged in locations that trace or reside within outsole outlines 21 corresponding to a pair of footwear. The outsole outlines 21 includes visual indicia or demarcations, for example, of a contrasting color to the pile 18, so that a user can readily see where to place his/her feet on the mat.

In the illustrated embodiment of FIG. 1, the mat includes at least eight UVC emitters 17 for each outsole outline, comprising at least three UVC emitters 17 located in a front portion 21F of the outsole, at least two UVC emitters 17 located in a heel portion 21H, and at least two UVC emitters 17 located in an arch portion 21A between the front portion and the heel portion. In some embodiments, the mat includes five (5) UVC emitters arranged with four in the corners of a vertical or “portrait” rectangle, and one in the center of the rectangle.

Each opening 20 through the pile 18 relative to the respective UVC emitter 17 is configured to provide a predetermined radiation pattern on the outsoles. In some embodiments, the predetermined radiation pattern includes multiple irradiated regions 22, such as illustrated in FIG. 2A. Notably, the predetermined radiation pattern is symmetrical above and below a horizontal midline so there is no distinguishable difference in the top and bottom halves of FIG. 2A. In some embodiments, as shown in FIG. 2B, the predetermined radiation pattern provides an azimuthal spread of about 60 degrees symmetrically about the z axis, for a total view angle spread of about 120 degrees. In that regard, the height of the pile can be configured to provide the predetermined radiation pattern, including the total view angle spread of about 120 degrees.

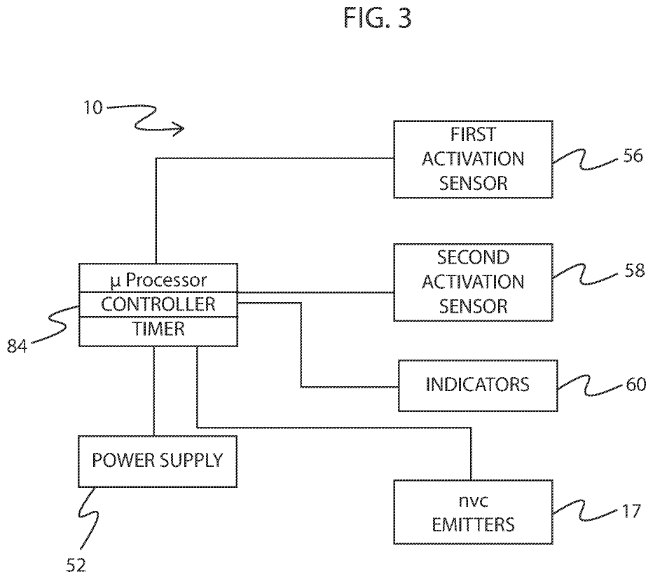

In some embodiments, as shown in FIG. 3, the integrated mat also includes a power supply 52, a first activation sensor 56 and a second activation sensor 58 configured to activate the plurality of UVC emitters 17, and at least one indicator 60 to provide a visual or audio signal to the user during activation of the UVC emitters 17. The first and second activation sensors 56 and 58 may be any combinations of the following sensors: (1) motion sensor, for example, infrared (IR) motion sensor, (2) proximity sensor, (3) pressure or weight sensor, and (4) light sensor, for example, photoresistor.

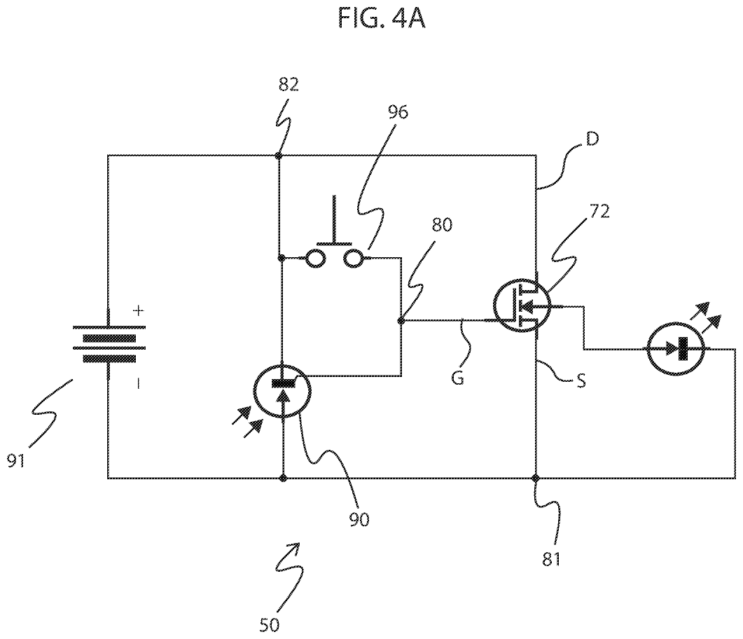

The power supply 52 provides power to components via an electrical circuit 50, an embodiment of which is shown in FIG. 4. In some embodiments, with reference to both FIG. 3 and FIG. 4, the power supply 52 is a DC power source, e.g., a battery (a single use battery, or a rechargeable battery 91). In some embodiments, the power supply 52 is an AC power source, for example, an electrical wall socket that is accessed via a connector plug. The electrical circuit 50 is configured such that power supplied to the UVC emitters 17 is condition on at least a first detection by the first activation sensor, for example, weight sensor 96, as a power-saving measure. In some embodiments, power supplied to the UVC emitters 17 is conditioned solely on a joint (but not necessarily simultaneous) occurrence of both the first detection by the first activation sensor 56, e.g., weight sensor 96, and a second detection by the second activation sensor 58, e.g., an IR motion sensor 90, so as to minimize the risk of a “false” triggering of either the first activation sensor or the second activation sensor.

In some embodiments, each or some of these activation sensors may be configured with a threshold setting as another measure to minimize the risk of a “false” trigger where the false trigger event is perpetrated by something other than an intended user, such as, for example, a bird, squirrel, dog or cat. With a motion sensor, the motion threshold may be a predetermined physical range of detected movement or motion and/or a predetermined proximity to the sensor. With a proximity sensor, the proximity sensor may be predetermined distance to the sensor. With a pressure or weight sensor, the weight threshold may be a minimum weight corresponding to a small child or a purposeful tapping or stamping of a foot. With a light sensor, the light threshold may be a minimum level of obscurity of light reaching the light sensor. In some embodiments, each of these thresholds may be adjusted to vary the sensitivity of the sensors in adapting to the physical environment in which the integrated mat is used.

As understood by one of ordinary skill in the art, the first and second activation sensors 56, 58 may be positioned anywhere on or in the mat 10 as desired or needed. In some embodiments, the IR motion sensor 90 as one of the activation sensors is positioned at a location on or near an outer edge 30 of the mat so as not to interfere with a user's ability to step onto the mat. It may occupy a housing 92 that is also housing the power supply 52. In some embodiments, a proximity sensor 94 as another of the activation sensors may also share the housing 92. In some embodiments, weight sensors 96 as further activation sensors are embedded in the mat 10, for example, between the first and second layers 12 and 14, or below the second layer 14, at locations within the outsole outlines 21 for detecting weight and thus presence of a user standing on the mat. In some embodiments, the photoresistors 98 as yet additional activator sensors each are located adjacent a respective center UVC emitter, within the respective outsole outlines 21, to detect changes in light or shadows cast by a user standing on the mat.

In some embodiments, the switch in the electrical circuit 50 includes a field-effect transistor (FET) switch 72 (N or P channel) with three terminals, namely, a source S, a drain D and a gate G. In some embodiments, the switch in the electrical circuit 50 includes a field-effect transistor (FET) switch 72 (N or P channel) with three terminals, namely, a source S, a drain D and a gate G. In the illustrated embodiment of FIG. 4A, the power supply is a rechargeable battery 91 and the switch is an N-channel configuration FET switch 72, where the first and second activation sensors are the IR motion sensor 90 and a weight (load) sensor 96, respectively, the gate G of the FET switch 72 is a junction 80 of the IR motion sensor 90 and weight sensor 96, the source S is a junction 81 of the IR motion sensor 90 and the UVC emitters 17, and the drain D is the weight sensor 96. As understood by one of ordinary skill in the art, the circuit is configured with the first and second activation sensors in parallel, where an FET switch 72 manages delivery of power to the UVC emitters 17 in response to activation of either the first or the second activation sensor.

In the illustrated embodiment of FIG. 4B, the power supply is a rechargeable battery 91 and the switch is an N-channel configuration FET switch 72, where the first and second activation sensors are the IR motion sensor 90 and a weight (load) sensor 96, respectively, and the gate G of the FET switch 72 is the weight or sensor 96, the source S is a junction 81 of the IR motion sensor 90 and the UVC emitters 17, and the drain D is a junction 82 of the IR motion sensor 90 and the battery 91. As understood by one of ordinary skill in the art, the circuit is configured with first and second activation sensors in series, where the FET switch 72 manages delivery of power to the UVC emitters 17 in response to activation of the first and second activation sensors, which for example, in the illustrated embodiment, are the IR motion sensor 90 and the weight sensor 96 respectively, so that a user must effectively trigger both the IR motion sensor 90 and the weight sensor 96 in order for the switch 72 to allow power delivery to the UVC emitters.

In some embodiments, no particular sequence of activation is required in order for activation of the UVC emitters. In some embodiments, a particular sequence of activation is required, for example, the motion sensor must be triggered first followed by the weight or light sensor. In some embodiments, the first and second activation sensors are required to be simultaneously triggered, or within a time window, in order for the activation of the UVC emitters. Embodiments of the present invention are not limited to the circuit diagrams of FIG. 4A and FIG. 4B.

The one or more indicators 60 of the integrated mat 10 serve to inform the user when the UVC emitters are actively in operation and radiating. In some embodiments, the indicators are visual indicators, for example, LEDs 97 that emit radiation in the visible spectrum to be seen by the user. As such, the indicators 60 are located in regions outside of the outsole outlines 21 so they are not obscured by the user's footwear when standing on the mat. In some embodiments, the mat includes first and second indicators, each of which corresponds with a respective outsole outline, for example, above the toe region of each outsole outline.

In some embodiments, the integrated mat includes a controller 84 with a microprocessor, as show in FIG. 3, configured to execute logic that controls charging cycles of the power supply 52 that includes a rechargeable battery and processes detection signals from the first and second activation sensors 56, 58 so as to determine duration of activation of the UVC emitters 17 and their emission intensity in sanitizing the outsoles of a user stepping on the integrated mat. The logic can also allow for improved power management extending the useful life of the UVC emitters 17 and the rechargeable battery.

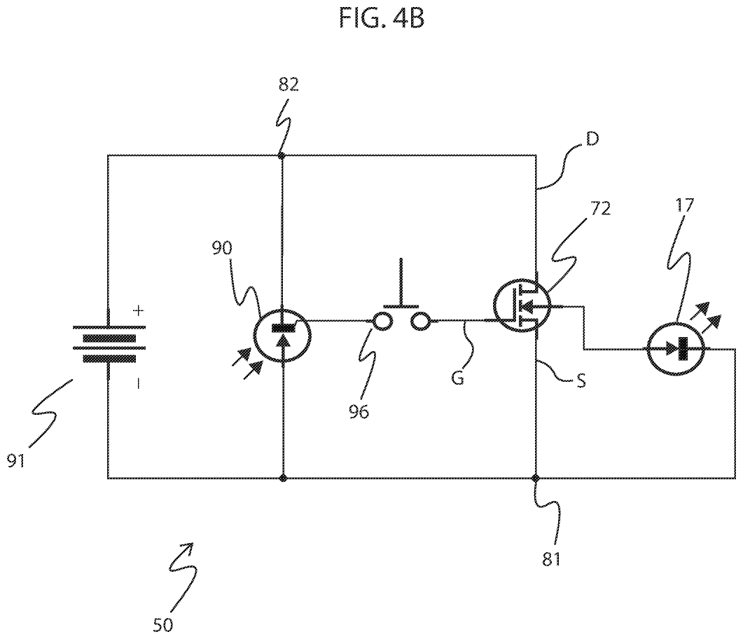

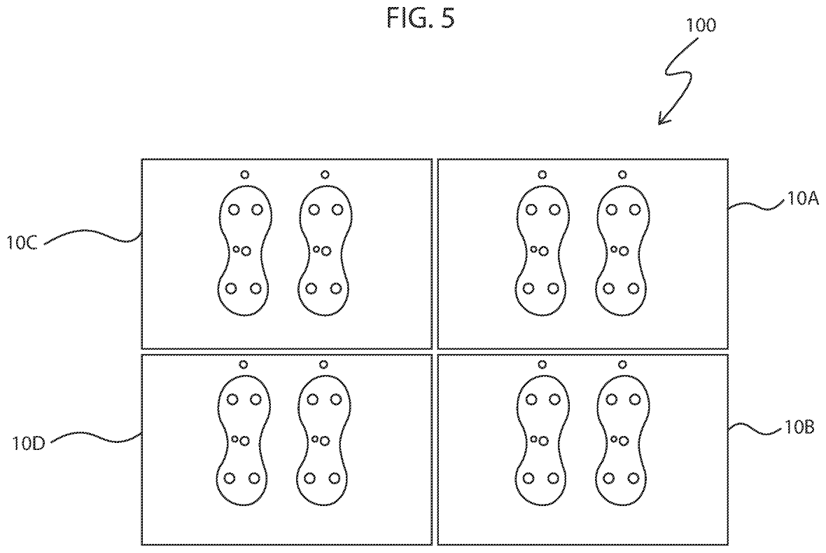

In some embodiments, as shown in FIG. 5, a sanitation row or array 100 is formed with a plurality of integrated mats 10A-10D, each configured as a module that can be coupled to and used with other modules to provide an expanded sanitation area upon which multiple users may step and stand on simultaneously for sanitation treatment of the outsoles of their shoes. In that regard, the mats are configured with fasteners or couplers 102 (see FIG. 1), e.g., hook and loop fasteners) along selected or each of outer edges 30. The resulting array can be formed to suit a variety of configurations of surface area for simultaneous use by multiple users for footwear outsole sanitation.

In some embodiments, each integrated mat 10i of the array 100 includes its respective single use or rechargeable battery. In some embodiments, one or more integrated mats of the array includes its respective single-use or rechargeable battery while other integrated mats are electrically coupled thereto. In some embodiments, one or more integrated mats are electrically connected to an electrical wall socket while other integrated mats are electrically connected thereto.

The controller 84 (see FIG. 3) is configured to receive data or signals output from the activation sensors and compare the signals to the thresholds (e.g., preset sensor parameters), and when the microprocessor determines that sensed values are within range as defined by the thresholds the controller outputs one or more signals to activate the UVC emitters 17. In some embodiments, as a parameter of irradiation, a duration of activation of the UVC emitters is predetermined and preset, where the controller utilizes a timer to control the duration of activation. As another parameter of irradiation, an intensity of emission by the UVC emitters is dependent on a distance determination or calculation by the motion or proximity sensor, where the distance is utilized by the microprocessor to generate a pulse width modulation output signal in controlling the voltage applied to the UVC emitters.

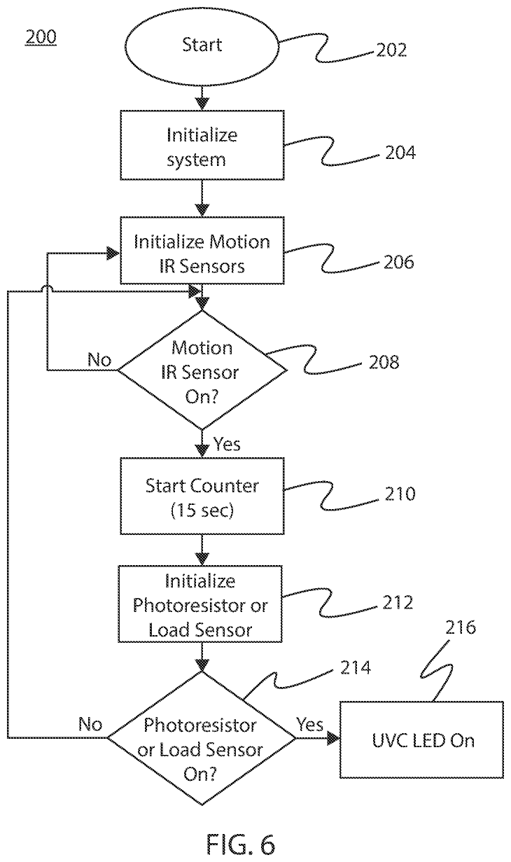

FIG. 6 shows a flowchart of acts performed by the controller, according to some embodiments. The acts begin at Start Block 202, followed by Process Block 204 where the system is initialized. At Process Block 206, the first activation sensor, e.g., IR motion sensor, is initialized, which is followed by Decision Block 208 with a query as to whether the IR motion sensor has been triggered. If no, the system returns to Process Block 206. If yes, the timer is started for a count of a predetermined duration, e.g., 15 seconds. The system then initializes the second activation sensor (e.g., the weight sensor or the light sensor), which is followed by Decision Block 214 with a query as to whether the second activation sensor has been triggered. If no, the system returns to the Decision Block 208. If yes, the system activates the UVC emitters.

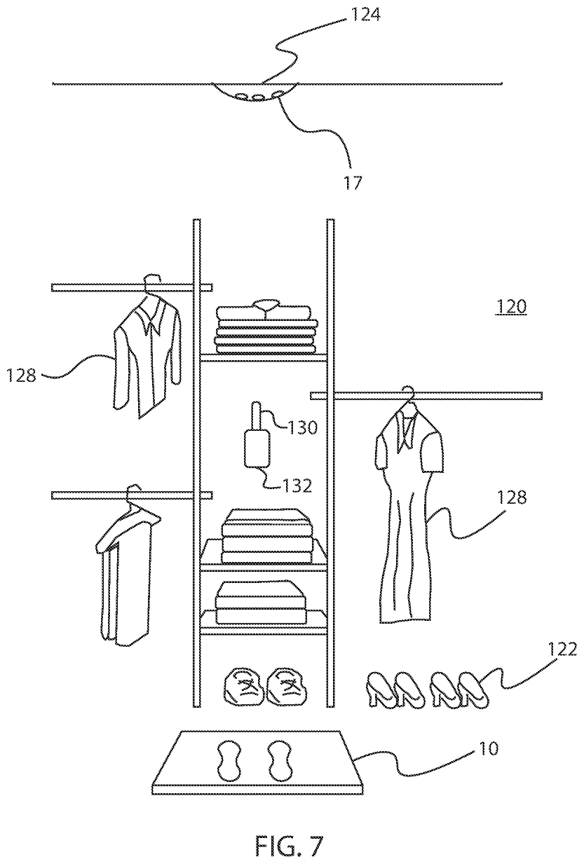

In some embodiments, as shown in FIG. 7, an apparel sanitation system 120 for use indoors, such as in a clothes closet, that includes at least one integrated mat 10 for sanitizing footwear 122 and a wall- or ceiling-mounted unit 124 with one or more UVC emitters 17 configured to sanitize apparel 128, including apparel hung on racks. In some embodiments, the system includes a hand-held sanitizing wand 130 that may be stored in a bracket or case 132 affixed to a wall and deployed for directed application and radiation of apparel, including hanging jackets, shirts, dresses, pants, coats, and the like.

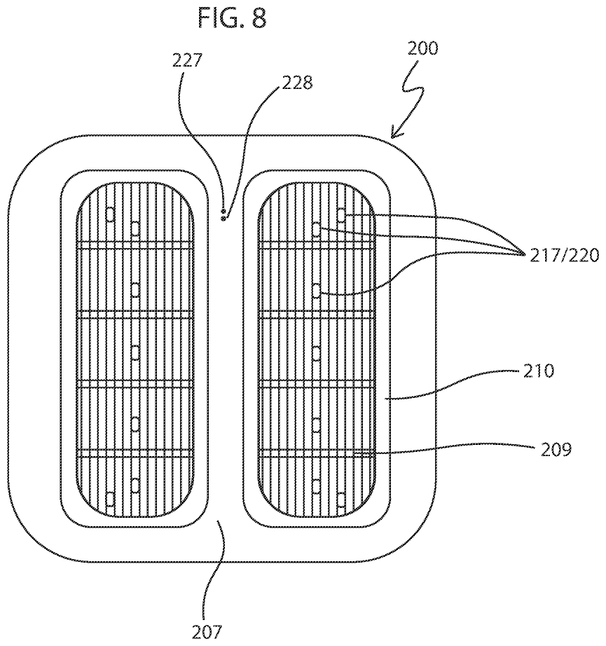

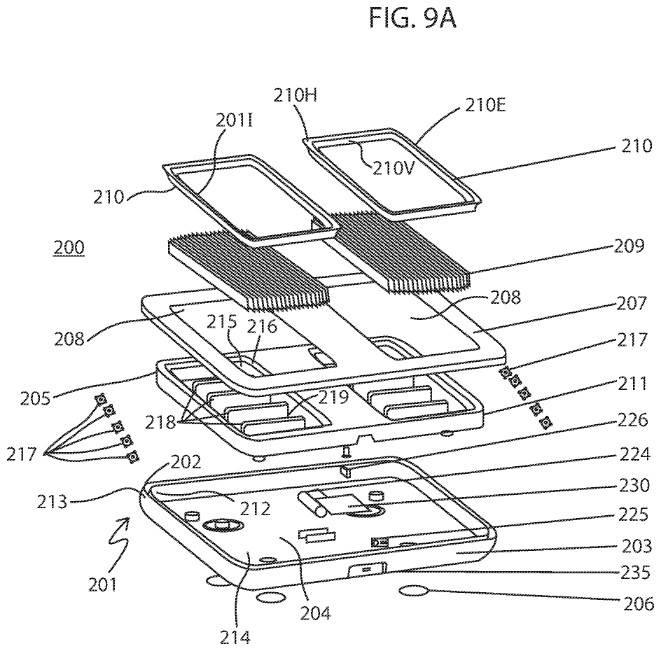

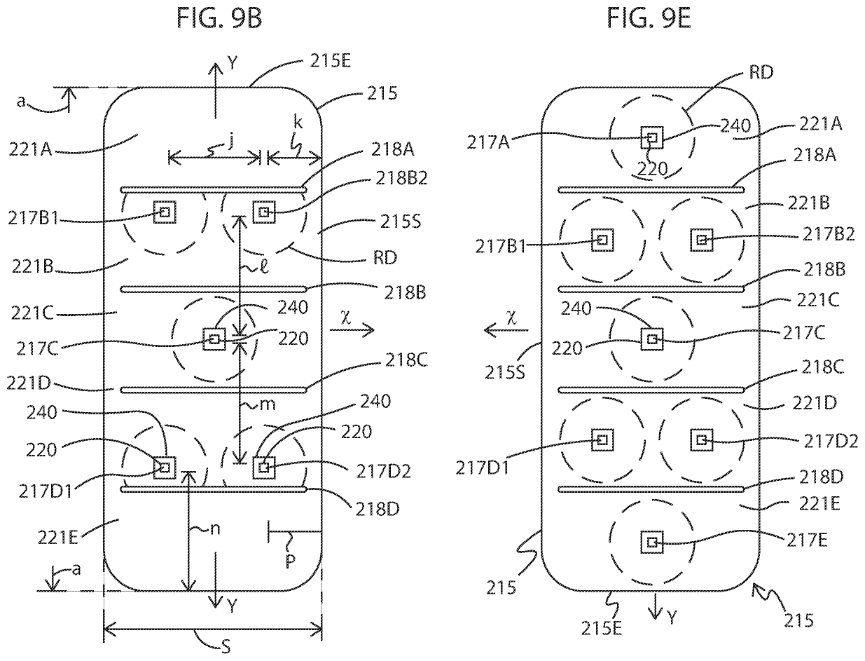

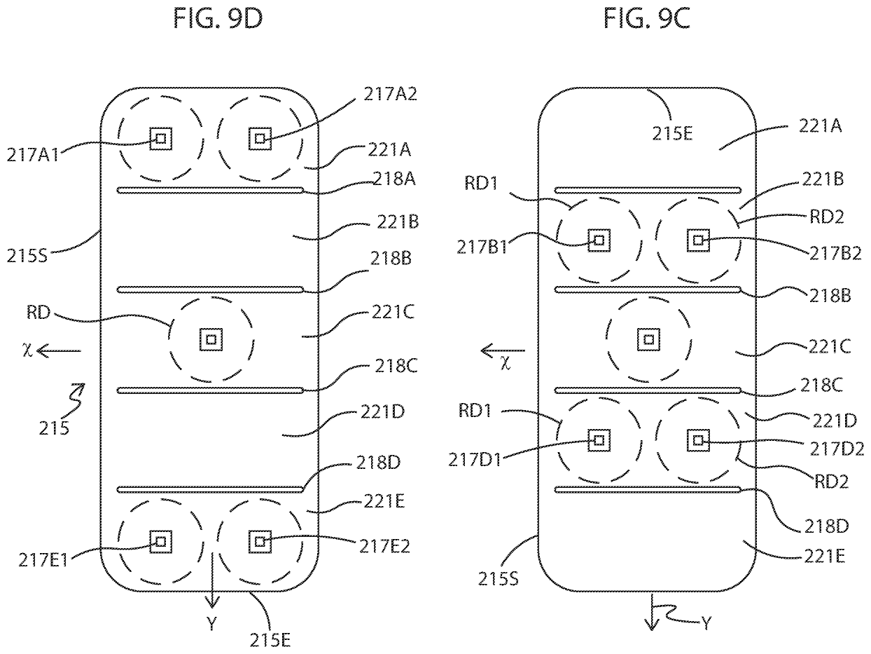

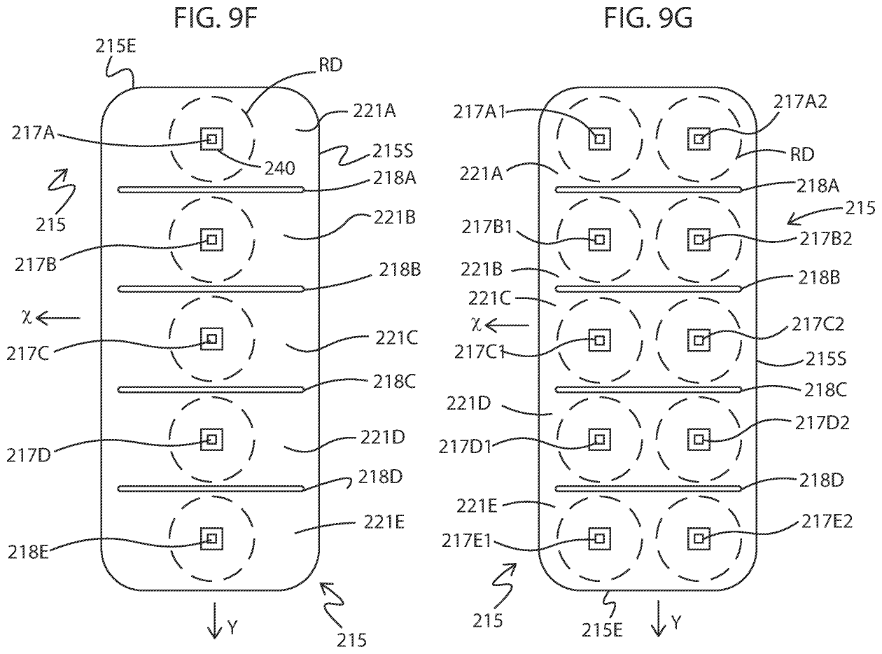

With reference to FIG. 8 and FIG. 9, a footwear sanitizing station 200 includes a generally-rigid housing 201 in which a plurality of UVC emitters 217 are housed to sanitize objects, including footwear, that are placed on the station 200. In some embodiments, the housing 201 includes a bottom member or cover 203 defining an interior 204 in which a base tray 205 sits to support an upper panel 207. The upper panel 207 has one or more cutout portions 208, each of which is configured to receive a respective platform grate 209 that is secured to the respective cutout portion 208 of the upper panel 207 by a respective edge guard 210 that traces the peripheries of the respective grate 209 and cutout portion 208. In the illustrated embodiment of FIG. 8, the station 200 includes two platform grates 209 that correspond to two cutout portions 208 in the upper panel 207, which are secured to the upper panel 207 by two edge guards 210, where each of the grate can receive and support a respective footwear whose sole is sanitized by UVC emitters 217 of the station 200. An exterior lower surface of the bottom cover 203 may include friction-inducing pads 206, for example, rubber pads, to help reduce movement or slippage between the station and the surface it is on.

In some embodiments, the upper panel 207 is constructed of an aluminum alloy material, the bottom cover 203 is constructed of plastic, and the edge guards 210 are constructed of a suitable plastic, for example, polypropylene. In some embodiments, the base tray 205 is constructed of injection-molded plastic that is configured with a peripheral overhang 211 that sits in a gap space 202 within an inner peripheral wall 212 and an outer peripheral wall 213, both of which extend generally perpendicularly, parallel with each other, from a base panel 214 of the bottom cover 203. In some embodiments, the upper panel 207 is constructed of glass, for example, stalinite glass which offers a very smooth surface, less porous compared to metals and plastics, which facilitates cleaning and sanitation in minimizing bacterial or germ growth. In some embodiments, the upper panel 207 is an optional component that is absent from the station 200.

For each grate 209, the base tray 205 is configured with a corresponding well 215 that surrounded by a recessed circumferential edge 216 to contact, support and help retain the grate 209 in the well 215. Within each well, the base tray 205 is configured with a plurality of raised ridges 218 that also contact and support the grate 209 in each well 215. The ridges extend in a direction generally parallel with a longitudinal axis of the station and generally divide each well into subwells 215S. The ridges are spaced evenly in the well such that each subwell is generally of similar shape and size where each subwell has a generally identical first dimension D1 along the longitudinal axis Y and a generally identical second dimension D2 along a lateral axis X generally perpendicular to the longitudinal axis Y.

A bottom 219 of each well 215 is configured with a plurality of apertures 220 each for a respective UVC emitter 217 to emit radiation into the well 215 and upwardly toward and through the grate 209 to reach objects placed on the grates 209, including footwear and their outsoles. In the illustrated embodiment, the station 200 includes ten UVC emitters 217 that are positioned throughout the well 215, with one or more in a respective region 221 defined between the ridges 218 which are generally equally spaced apart in the well 215, so that each UVC emitter generally radiates a respective portion of the object on the grates 209, for example, the footwear.

In that regard, parameters of the well 215 and the ridges 218, including, for example, the size of the aperture, the depth D of the well, the size of the region (subwell) 221, the layout, plurality and height of the ridges 218, the thickness of the grate 209, the spacing between slats of the grate 209, can affect the amount of radiation from the UVC emitters that reach the outsole, and the pattern of radiation on the outsole.

Each edge guard 210 is configured with a vertical portion 210V and a horizontal portion 210H. The vertical portion 210V surrounds the outer circumference of the grate 209 while also sitting within the cutout portion 208 of the upper panel 207. The horizontal portion 210H has an outer edge 210E that is outside of or greater than the cutout portion and an inner edge 2101 that is inside of or lesser than the grate so that the horizontal portion 210H bridges and covers any gaps between the grate 209 and the cutout portion 208 and also retains the grate in the well 215.

In FIG. 9B, a base panel 214 configured with a predetermined UVC emitter configuration is shown for UVC emitters, each with a sanitation/germicidal radiation diameter RD of about 50-60 mm. In particular, the apertures 220 and corresponding UVC emitters 217 are arranged in a nonlinear or alternating pattern (e.g., 2-1-2 pattern), having a center subwell 221C (with a single, generally centered UVC emitter 217C) situated between subwell 221B with and subwell 221D (each of which has two side-by-side UVC emitters 21761. 21762 and 217D1. 217D2). Two end subwells 221A, 221E are without any UVC emitters. Parameters including configuration measurements in the illustrated embodiment of FIG. 9B are listed below:
a=323.58 mm
j=57.29 mm
k=29.4 mm
l=76.4 mm
m=76.4 mm
n=78.65 mm
p=29.4 mm
s=125.08 mm

In some embodiments of the nonlinear or alternating pattern, as shown in FIG. 9C, the pairs of UVC emitters 217B, 217D are situated closer to the ridges 218B and 218C, respectively, so that interference between the ridges and the corresponding incidental radiation diameters RD is minimized. The UVC emitters 217B and 217D may be positioned generally equidistanced along the longitudinal axis Y between adjacent ridges. Notably, each of the subwells 221B and 221D, each with two side-by-side UVC emitters 217B and 217D, respectively, presents a wider (twice as wide) incidental radiation spread (RD1+RD2) that may be better suited for irradiation of the wider ball and heel regions of footwear. In contrast, the subwell 221C with the centrally-located single UVC emitter 217C may be better suited for irradiation of the narrower arch region of footwear.

In some embodiments of the alternating pattern of 2-1-2, as shown in FIG. 9D, the subwells 221B and 221D are devoid of UVC emitters, but the end subwells 221A and 21E, each has two side-by-side UVC emitters 217A1, 217A2 and 217E1, 217E2 which are positioned generally equidistanced along the longitudinal axis Y between the end walls 215E of the well 215 and the adjacent ridges 218A, 218E, respectively.

In some embodiments, an alternating pattern of 1-2-1-2-1, as shown in FIG. 9E, has each subwell having at least one UVC emitter. In particular, end subwells 221A and 21E and subwell 221C each has one UVC emitter 217A, 217E and 217C respectively, whereas subwells 221B and 221C each has two, side-by-side UVC emitters 21761, 21762 and 217C1, 217C2.

In FIG. 9F, another predetermined UVC emitter configuration includes the apertures 220 and UVC emitters 217 arranged in a linear or nonalternating pattern (e.g., 1-1-1-1-1 or 1×n pattern for n plurality of UVC emitters, each also having a sanitation/germicidal radiation diameter RD of about 50-60 mm). As illustrated, each subwell has a single, centrally-located UVC emitter, where all the UVC emitters are all aligned along a common centered longitudinal axis generally parallel to the longitudinal axis Y. Parameters including configuration measurements in the illustrated embodiment of FIG. 9F are listed below:
a=323.58 mm
b=60.04 mm
c=30.54 mm
d=60.04 mm
e=28.24 mm
f=28.24 mm
g=28.24 mm
h=28.24 mm
i=30.54 mm
s=125.08 mm

Notably, with placement of the apertures 220 and corresponding UVC emitters 217 generally in the center of each subwell, where both the first and second dimensions D1, D2 of the subwell are not less than the incidental radiation diameter RD of the UVC emitters, there is minimal, if any, interference between the ridges 218, the sidewall and/or end wall of the wells 215, and the incidental radiation diameter RDs in each subwell. That is, there is minimal, if any, shadowing created by the ridges 218, and most of the radiation emitted by the UVC emitters efficiently reaches the grate above each well.

In some embodiment, another predetermined UVC emitter configuration, as shown in FIG. G, includes the apertures 220 and UVC emitters 217 arranged in a nonalternating pattern (e.g., 2-2-2-2-2 or 2×n pattern for 2n plurality of UVC emitters, each also having a sanitation/germicidal radiation diameter RD of about 50-60 mm). Each subwell includes two, side-by-side UVC emitters, with the first UVC emitters of each subwell in longitudinal alignment with each other and the second UVC emitters of each subwell in longitudinal alignment with each other.

Thus, depending on the parameters, manufacturing and operational costs, footwear irradiation coverage and energy usage can be optimized. In some embodiments, configuring each well 215 with the gap spacing between adjacent ridges 218 to be generally equal to the UVC emitter's incidental radiation diameter optimizes load distribution and radiation diameter. It is understood that the incidental radiation diameter of the UVC emitter is adjustable within limits and that the UVC emitter inherently has a radiation cone of about 120 degrees. Thus, the incidental radiation diameter takes into account an elevational spacing between the UVC emitter and the incidental surface as well as the radiation cone of the UVC emitter and shadowing resulting from the ridges 218.

In some embodiments, each subwell 221 separated from adjacent subwell(s) by a ridge 218 is generally rectangular in being defined by a first dimension D1 along the longitudinal axis Y and a second dimension D2 along the lateral axis X generally perpendicular to the longitudinal axis Y, where the first dimension D1 is about equal to the incidental irradiation diameter RD of each UVC emitter.

It is understood that the UVC emitter patterns are not limited to those illustrated herein and that different patterns provide different degrees of radiation coverage or irradiation efficiency.

In the illustrated embodiments, each well 215 has a length of about 13″, a width of about 5.0″ and a depth about 1.25″. Each ridge 218 in the well has a height about 1.11″, a width of about 0.8″ and a length of about 4.2″. Each well at its bottom wall also includes at least one drainage opening 230 that communicates with a drainage pathway to allow fluids collected in the well, including those that drip from the footwear on the grate 209, to exit the well and the station 200. Each grate has longitudinal bars and lateral bars. The longitudinal bars are about 0.1″ in diameter and separated from adjacent bars by a gap space of about 0.3″ which minimizes shadowing and adverse impact on effectiveness of sanitizing irradiation from the UVC emitters.

Notably, in some embodiments, the bottom wall of the well is formed with a raised mound 240 surrounding each UVC emitter 217 so that the UVC emitter is elevated above the bottom wall and thus elevated and removed from any fluid that may be collected at the bottom of the well. In some embodiments, the mount has a generally rectangular, e.g., square, shape around each UVC emitter.

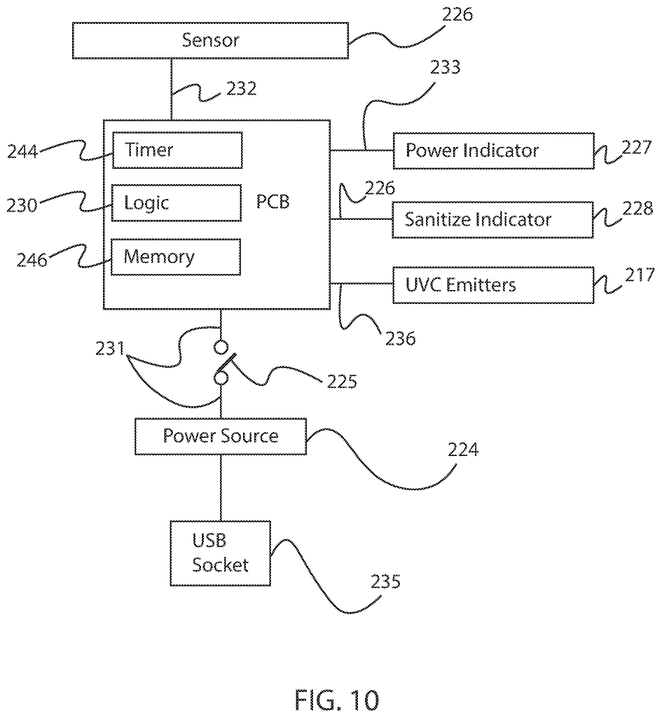

With reference to FIG. 10, the station 200 further includes components of an electrical system S that are housed in the housing 201, including a power source 224, a power switch 225, a printed circuit board (“PCB”) 230, a sensor 226, a power-on or stand-by indicator 227, a sanitize indicator 228, in addition to the UVC emitters 217.

In some embodiments, the PCB 230 and the power source 224 are connected via connection 231 and power is delivered to the PCB 230 upon actuation of the power switch 225 by the user. The power switch 225 is connected to the connection 231 via connection 24. Power is supplied to other components of the system S through the PCB 230 via other connections, including connection 232 with the sensor 226, connection 233 with the power indicator 227, connection 234 with the sanitize indicator 228, and connection 236 to the UVC emitters 217. It is understood that these connections may be any suitable connections, including, wire and/or wireless.

In some embodiments, the power source 224 includes a rechargeable battery, e.g., an 18650 lithium ion battery of 3.7V and capacity of 2000 mAh, with charging protection, with which the PCB30 is compatible. The battery may be rechargeable via, e.g., a micro USB socket 235 situated in the bottom cover 203. As understood, DC power can be delivered to the rechargeable battery via a charging unit with a USB port, and a cable with a USB connector at one end for coupling with the USB port, and a micro USB connector for coupling with the micro USB socket 235.

In some embodiments, the PCB 230 is configured to operate in at least dual modes, e.g., a Stand-By Mode and a Sanitize Mode after a user has activated the power switch 225 to deliver power to the PCB. Once powered, the PCB operates in the Stand-By Mode by delivering power to the actuator 226 and the power indicator 227. In some embodiments, the power indicator 227 includes an LED to provide a visual signal to a user that the station 200 is powered and ready to sanitize footwear, and the actuator 226 includes a pressure or touch sensor configured to detect a threshold load on the one or more platform grates 209, such as when footwear is placed on the one or more platform grates.

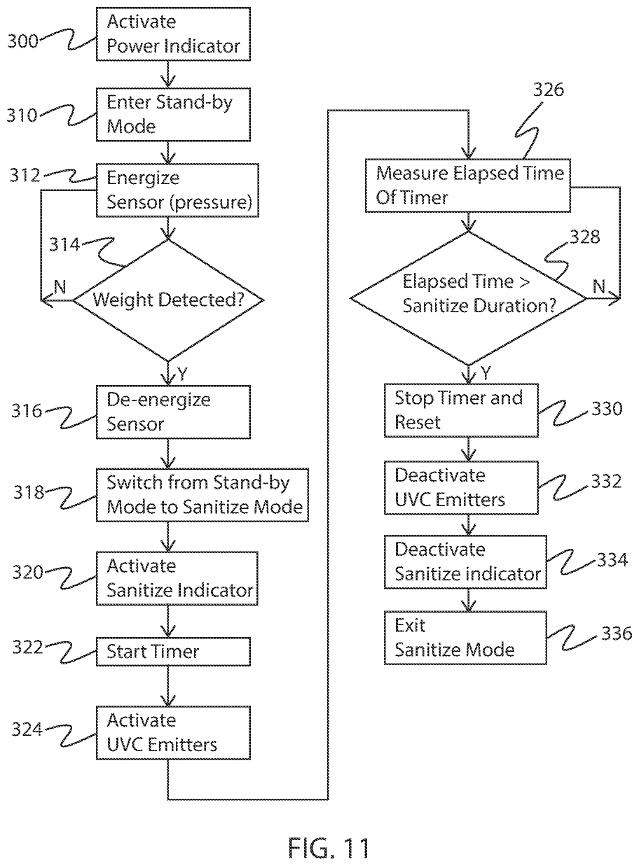

In some embodiments, control and operation of the electrical components are implemented by a controller 240 comprising a processing unit 242, e.g., a picocontroller, communicating with a timer 244 and a memory 246, wherein is stored software for operation of system 200, as shown in FIG. 10. FIG. 11 illustrates a flow chart of a process executed by the controller, according to some embodiments, for controlling and operating the station S in sanitizing footwear. Upon a user activating the power switch 225, the process executed by the controller 240 may begin. At Block 310, the controller activates the power indicator 227 to provide a cue to the user that the station 200 is energized and ready to begin the sanitation process. In some embodiments, the power indicator 227 includes an LED that is positioned in or on the upper panel 207, for example, between the two platform grates 209, to provide a visual cue to the user.

At Block 310, the controller enters the Stand-By Mode to await detection of weight, including at least footwear, if not also the user wearing the footwear, on the one or more platform grates 209. At Block 312, the controller energizes the sensor 226. In some embodiments, the sensor 226 includes a pressure sensor that is responsive to the movement of the one or more platform grates 209 or otherwise configured to detect the presence of weight thereon. At Decision Block 314, if no weight has been detected by the sensor, the process returns to Block 312 and continues to energize the sensor 226. If weight has been detected by the sensor, the process advances to Block 316 where the controller 240 de-energizes the sensor 226 to save power, and at Block 318, switches from the Stand-By Mode to the Sanitize Mode.

At Block 320, the controller operating in the Sanitize Mode activates the sanitize indicator 228 to provide a cue to the user that the station 200 is ready to activate the UVC emitters 217. In some embodiments, the sanitize indicator includes an LED that is positioned adjacent the power indicator 227 between the two platform grates 209, to provide a visual cue to the user. The sanitize indicator 227 may also include a sound emitter to provide an audio cue during activation of the UVC emitters.

At Block 322, the controller starts a timer to time the Sanitize Mode and the activation of the UVC emitters to control the duration of the Sanitize Mode. At Block 324, the UVC emitters are activated for radiating the footwear on the platform grates 209. At Block 326, the time elapsed is determined, and at Decision Block 328 the time elapsed is compared to a Sanitize Duration. In some embodiments, the Sanitize Duration is about 30 seconds, but it can be set to any suitable or desirable duration of lesser or greater than 30 seconds. In Decision Block 328, if the time elapsed is not greater than the Sanitize Duration, the process returns to Block 326. If the time elapsed is greater than the Sanitize Duration, the Sanitize Mode has reached its end and the process advances to Block 330 wherein the controller stops the timer and resets to zero. Then in Block 332, the controller deactivates the UVC emitters, and in Block 334 deactivates the sanitize indicator 228 to signal to the user that the UVC radiation has terminated. In Block 336, the controller exits the Sanitize Mode. The user may then deactivate the power switch 225.

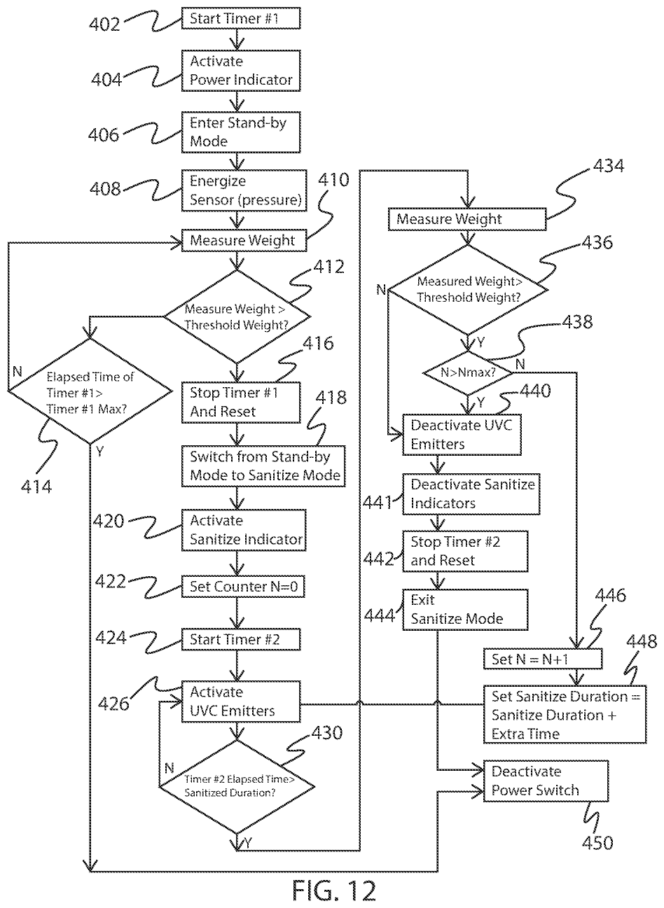

FIG. 12 illustrates a flow chart of a process executed by the controller, according other embodiments, for controlling and operating the station S in sanitizing footwear. Upon a user activating the power switch 225, the process executed by the controller 240, at Block 402, initiates timing by Timer #1, and at Block 404, the controller activates the power indicator 227. At Block 406, the station 200 enters the Stand-By Mode, and at Block 408, energizes the sensor 226.

At Block 410, a measured weight on the one or more platform grates 209 is determined, and at Decision Block 412, the measured weight is compared to a predetermined Threshold Weight. If the Measured Weight is not greater than the Threshold Weight, the process advances to Decision Block 422 where if an elapsed time of Timer #1 is not greater than a predetermined Threshold Timer #1 Max, the process returns to Block 410, for example, so as to allow more time for the user to more properly set the footwear on the platform grates 209. If the Elapsed Time of Timer #1 is greater than Threshold Time #1 Max, the process advances to Terminal 458 with deactivation of the power switch 225. The use of the predetermined Threshold Weight in the process is intended to prevent the activation of the UVC emitters unless a minimum weight is detected by the sensor 226.

If at Decision Block 412 the measured weight is greater than the Threshold Weight, the process advances to Block 416 where the Timer #1 is stopped and reset to zero, and at Block 418, the controller switches from the Stand-by Mode to the Sanitize Mode. At Block 420, the sanitizer indicator 228 is activated. At Block 422, a Counter N is set at zero. At Block 424, Timer #2 is initiated. At Block 426, the UVC emitters 217 are activated to radiate the footwear on the platform grates 209.

At Decision Block 430, if the elapsed time on Timer #2 is not greater than a predetermined Sanitation Duration, the process returns to Block 426. If the Elapsed Time on Timer #2 is greater than the predetermined Sanitation Duration, the process advances to Block 434 where weight on the platform grates 209 is again measured. At Decision Block 436, the measured weight is compared to the predetermined Threshold Weight to determine if the user has stepped off or removed the footwear from the platform grates 209. If the measured weight is not greater than the predetermined Threshold Weight, then presumably the user has in fact stepped off or removed the footwear from the platform grates, and the process continues to Block 440 where the UVC emitters are deactivated and to Block 441 where the sanitize indicator is deactivated. At Block 442, the Timer #2 is stopped and reset to zero. At Block 444, controller exits the Sanitize Mode. At Block 450, the controller deactivates the power switch.

However, if at Decision Block 436, the Measured Weight is greater than the Threshold Weight, then presumably the user has remained on the platform grates and desires an additional cycle of sanitation treatment where perhaps he repositions his footwear for further UVC radiation, the process advances to Decision Block 438 where if the count N is not greater than a predetermined Nmax (the maximum number of UVC radiation cycles permitted per Sanitation Mode), the process advances to Block 446 where the counter N is increased by one, and to Block 448 where the Sanitation Duration (UVC activation time period) is increased by an Extra Time, e.g., 20 seconds, and the process returns to Block 426.

If at Decision Block 438, the count N is greater than the Nmax, that is, where the user has exceeded the maximum number of radiation cycles permitted per Sanitation Mode, the process continues to Block 440.

In some embodiments, the power switch 225 is configured to be activated by a sensor that is responsive to voice, contact and/or pressure to energize the PCB 230. This sensor may be the sensor 226 in lieu of the power switch 225, or this sensor may be an additional sensor.

It is understood that the various acts of the processes of FIG. 11 and FIG. 12 need not occur in series but may occur in parallel and/or in different sequences than those depicted FIG. 11 and FIG. 12, and that any act of any one process may be incorporated in lieu of or in addition to any other acts of the processes described herein.

In some embodiments, the process deactivates the UVC emitters before deactivating the sanitize indicator as a safety measure so that the user is not given a cue indicating deactivation of the sanitize mode until the UVC emitters have been deactivated.

In some embodiments, the UVC emitters of the station 200 includes UVC LEDs that have a total power of 3 W, with each LED having 10,000 hours of actual usage time which is approximately 4 years, and the power source 224 includes a lithium ion rechargeable battery with a voltage of 3.7V and a capacity of 4000 mAh, which can be recharged via a USB cable, by a power source with 5V DC [is this AC or DC?], 2 A with a charge time of 6 hours. As such, the station on a full charge can operate about 500 cycles (30 seconds per sanitation cycle) in Sanitize Mode or [this is “or,” not “and,” right?] in Stand-By Mode for about 30 days. The station may have dimensions of about 41 cm×41 cm×6 cm (about 16.1″×16.1″×2.4″) or dimensions of 36 cm×36 cm×36 cm (about 14.2″×14.2″×2.4″).

With reference to FIG. 10, in some embodiments, a user begins operation of the station 200 by activating the power switch 225 which closes the connection 231 and allows power to be provided to the PCB 230. The power indicator 227 is activated to give the user a cue that the station is in Stand-By Mode, ready to receive objects, including footwear, on the platform grates 209. The user may then place, for example, one shoe on each platform grate 209, the weight of which is detected by the sensor 226 which in turn triggers the station to switch from Stand-By Mode to Sanitize Mode. The sanitize indicator 228 is activated to give the user a cue that the UVC radiation will begin. UVC emitters are then energized for a predetermined duration, for example, 30 seconds. After expiration of the predetermined sanitation duration, the UVC emitters are de-energized and the sanitize indicator are deactivated to give the user a cue that the UVC radiation has ended. The user may then safely remove the footwear from the platform grates 209. The station may then return to Stand-by Mode and either automatically deactivate the power switch or waits for the user to deactivate the power switch.

In some embodiments, the power indicator 227 provides a cue to the user (for example, continuous flashing) when the battery power source is low. The user may charge the battery by using a micro USB charge cable to connect the micro USB socket 235 to a mobile phone charging USB adapter configured for DC 5V, 2 more amps. The battery is configured to reach full charge in about 6 hours where the full charge holds for about 30 days.

In the preceding description, for the purposes of explanation, numerous specific details are set forth to provide a thorough understanding of various embodiments. It is apparent, however, that various embodiments may be practiced without these specific details or with one or more equivalent arrangements. In other instances, well-known structures and devices are shown in block diagram form in order to avoid unnecessarily obscuring various embodiments.

Various embodiments are described herein with reference to sectional illustrations that are schematic illustrations of embodiments and/or intermediate structures. As such, variations from the shapes of the illustrations as a result, for example, of manufacturing techniques and/or tolerances, are to be expected. Further, specific structural or functional descriptions disclosed herein are merely illustrative for the purpose of describing embodiments according to the concept of the present disclosure. Thus, embodiments disclosed herein should not be construed as limited to the particular illustrated shapes of regions, but are to include deviations in shapes that result from, for instance, manufacturing. Thus, the regions illustrated in the drawings are schematic in nature and their shapes are not intended to illustrate the actual shape of a region of a device and are not intended to be limiting. Moreover, it is also understood that the drawings are not necessarily to scale.

The foregoing is illustrative of example embodiments, and is not to be construed as limiting thereof. Although a few example embodiments have been described, those skilled in the art will readily appreciate that many modifications are possible in the example embodiments without materially departing from the novel teachings and advantages of example embodiments. Accordingly, all such modifications are intended to be included within the scope of example embodiments as defined in the claims. In the claims, means-plus-function clauses are intended to cover the structures described herein as performing the recited function and not only structural equivalents but also equivalent structures. Therefore, it is to be understood that the foregoing is illustrative of example embodiments and is not to be construed as limited to the specific embodiments disclosed, and that modifications to the disclosed example embodiments, as well as other example embodiments, are intended to be included within the scope of the present invention.

What is claimed is:

1. A footwear sanitation station comprising:
a housing with at least one grate configured to support footwear;
a base tray configured to support the at least one grate;
a plurality of UVC emitters configured to radiate the footwear supported on the at least one grate
a sensor;
a power indicator;
a sanitize indicator;
a power source; and
a controller with a processor configured to:
operate the station in two modes, including a Stand-By Mode and a Sanitize Mode; and
activate the power indicator and the sanitize indicator to provide cues to a user of the Mode of operation.

2. The footwear sanitation station of claim 1, wherein the sensor includes a pressure sensor configured to detect the presence of an object on the station by a weight of the object on the platform grate.

3. The footwear sanitation station of claim 1, wherein the power indicator includes a light indicator configured to provide a visual cue to the user of operation of the station in the Stand-By Mode.

4. The footwear sanitation station of claim 1, further including a power switch.

5. The footwear sanitation station of claim 1, further including an edge guard configured to extend around a periphery of a respective grate.

6. The footwear sanitation station of claim 1, wherein the base tray includes a well configured to receive a respective grate.

7. The footwear sanitation station of claim 6, wherein each well has a plurality of ridges configured to support the grate.

8. The footwear sanitation station of claim 6, wherein each well has a predetermined depth.

9. The footwear sanitation station of claim 1, wherein the processor is configured to energize the UVC emitters where the weight detected by the sensor exceeds a predetermined threshold weight.

10. The footwear sanitation station of claim 1, wherein the processor is configured to activate the power indicator and the sanitizer indicator in a predetermined sequence upon activation of the UVC emitters

11. The footwear sanitation station of claim 1, wherein the processor is configured to extend a time duration of operation of the Sanitize Mode.

12. The footwear sanitation station of claim 1, wherein the power source is rechargeable.

13. A footwear sanitation device comprising:
a base tray with at least one well, each well having at least one ridge;
at least one grate each configured to be received in a respective well and supported by the ridge in the respective well; and
at least one UVC emitter situated in a respective well and each configured to direct its radiation toward the grate.

14. The footwear sanitation device of claim 13, wherein each well includes subwell, each of which includes at least one UVC emitter.

15. The footwear sanitation device of claim 13, wherein each well includes a plurality of UVC emitters arranged in an alternating pattern.

16. The footwear sanitation device of claim 15, wherein the alternating pattern includes a 2-1-2 pattern.

17. The footwear sanitation device of claim 15, wherein the alternating pattern includes 1-2-1-2-1 pattern.

18. The footwear sanitation device of claim 14, wherein each well includes a plurality of UVC emitters arranged in a nonalternating pattern.

19. The footwear sanitation device of claim 18, wherein the nonalternating pattern includes 1-1-1-1-1 pattern.

20. The footwear sanitation device of claim 18, wherein the the nonalternating pattern includes 2-2-2-2-2 pattern.
